(19) United States
(12) Patent Application Publication
(10) Pub. No.: US 2020/0289410 A1
(43) Pub. Date: Sep. 17, 2020

(54) CAPSULE ENDOSCOPY DELIVERY

(71) Applicant: ColonaryConcepts LLC

(72) Inventors: Corey A. SIEGEL, Palm Beach Gardens, FL (US); Michael K. ALLIO, Palm Beach Gardens, FL (US)

(21) Appl. No.: 16/084,174

(22) PCT Filed: Mar. 28, 2017
(86) PCT No.: PCT/US2017/024620

§ 371 Date: Sep. 11, 2018

(51) Int. Cl. A61K 9/00; A61K 31/047; A61B 1/04; A23L 33/00

(52) U.S. Cl. CPC A61K 9/0053; A61K 31/047; A23V 2002/00; A23L 33/40; A61B 1/041

(57) ABSTRACT

Described herein are methods and compounds for aiding in the performance of capsule endoscopy on an individual. The efficacy of the capsule endoscopy procedure is limited in at least in part by the power provided by the battery within the capsule. The methods and compounds described herein provide improved capsule endoscopies yielding valuable clinical data. In addition, the methods and compounds described herein provide for a more satisfying experience for the individual undergoing a bowel preparation related to a capsule endoscopy and thus increase compliance with the bowel preparation.

### CROSS-REFERENCE

This application claims the benefit of U.S. Provisional Application No. 62/314,897, filed Mar. 29, 2016, which is incorporated herein by reference in its entirety.

### BACKGROUND

Capsule endoscopy is a technique for visualizing the GI tract of an individual. Capsule endoscopy is used in particular to visualize the small and/or large bowel of an individual for areas of the small bowel that are not visualized with other forms of endoscopy and as a non-invasive method for polyps in the colon.

The capsule used for capsule endoscopy contains a camera that periodically takes photographs of the intestines and transmits them to a wireless receiver. The capsule is also typically configured to transmit a signal from which its relative location within the bowel of an individual may be determined.

Capsule endoscopy is useful in diagnosing certain abnormalities of the small and large bowel such as Crohn's disease, GI blead, polyps, and ulceration.

### SUMMARY

Described herein are compositions, methods, and kits for promoting more efficient and more productive endoscopic procedures and in particular capsule endoscopy and colonoscopy procedures (especially colonoscopies following capsule endoscopies).

### Improved Efficiency

Capsules used in capsule endoscopy procedures are typically very small in size, having dimensions on the order of tens of millimeters. Capsules used in capsule endoscopy typically include a camera, a wireless transmitter, a battery, as well as additional electronic components. The amount of battery power that is available in a battery small enough to fit into a capsule typically limits the amount of images that may be generated and transmitted by the capsule during an endoscopy procedure. As such, the images are not typically taken in a continuous fashion but rather intermittently so that an entire contiguous length of a region of the gastrointestinal tract is not typically visualized. As such, lesions are frequently missed in capsule endoscopy when a region having a lesion in it is not imaged. A capsule endoscopy procedure becomes much more clinically valuable if images are taken more frequently. Taking and transmitting images more frequently is achieved with optimization of battery usage.

Described herein are compositions, methods, and kits for optimizing battery usage in capsule endoscopy and thus for providing more clinically valuable capsule endoscopy procedures by: 1) providing targeted delivery of the capsule to a specific region within the gastrointestinal tract, and/or 2) promoting increased motility within the gastrointestinal tract that acts to propel the capsule more rapidly through the gastrointestinal tract.

### More Productive Endoscopic Procedures

In certain circumstances a capsule endoscopy is followed by the performance of a colonoscopy procedure. Typically, an individual undergoing a colonoscopy following a capsule endoscopy must ingest an additional bowel preparation in addition to the bowel preparation that the individual ingested before the capsule endoscopy. Each bowel preparation that an individual must ingest is very challenging for most individuals, who will suffer from hunger pangs, nausea, fatigue—and therefore the likelihood of complete compliance with a second bowel preparation regimen is very low because completing two bowel preparation regimens in a short period of time is challenging for most individuals thus compliance is even lower than the already low compliance associated with a single bowel preparation regimen. Poor compliance with bowel preparation regimens is associated with poor evacuation of stool from the colon which leads to poor visualization during colonoscopy. Poor visualization is associated with missed lesions, because retained stool in the bowel covers or otherwise prevents visualization of lesions of the bowel wall.

Described herein are compositions, methods, and kits for providing a bowel preparation to be used by an individual in between a capsule endoscopy and a colonoscopy that increases the satisfaction and compliance of the individual with the two bowel preparation regimens.

Described herein is a method for delivering a capsule comprising a camera to the gastrointestinal tract of an individual in order to perform a capsule endoscopy procedure, comprising: providing a delivery vehicle to the individual to ingest with the capsule, wherein the delivery vehicle comprises a food item; and performing the capsule endoscopy procedure after the individual has ingested the capsule and the delivery vehicle. In some embodiments, the food item comprises a solid food. In some embodiments, the delivery vehicle further comprises a bowel motility promoting agent, and wherein the bowel motility promoting agent is incorporated with the food item. In some embodiments, the food item comprises one or more food ingredients mixed together with the bowel motility promoting agent. In some embodiments, one or more food items comprise a kit, wherein the one or more food items are modular elements of the kit.

In some embodiments, the modular elements of the kit are ingested by an individual in a sequence after the individual has ingested the capsule. In this way, the sequentially ingested food items provided a sequential peristalsis effect that propels the capsule through the gastrointestinal tract. In some embodiments, the individual is instructed to ingest the modular elements of the kit in a specific order. In some embodiments, the individual is instructed to ingest the modular elements of the kit within a certain time period overall, and modular elements within the kit at certain specific time periods. In some embodiments, the bowel motility promoting agent comprises a laxative. In some embodiments, the bowel motility promoting agent comprises one or more of Benzamide, Cisapride, Domperidone, Erythromycin, Itopride, Mosapride, Metoclopramide, Prucalopride, Renzapride, Tegaserod, Mitemcinal, Levosulpiride, and Cinitapride. In some embodiments, performing the capsule endoscopy procedure occurs within one hour after the individual has ingested the capsule and the delivery vehicle. In some embodiments, the method comprises instructing the individual to ingest the capsule first and the delivery vehicle second. In some embodiments, the method comprises instructing the individual to ingest the delivery vehicle within 15 minutes of ingesting the capsule. In some embodiments, the method comprises instructing the individual to ingest the delivery vehicle first and the capsule second. In some embodiments, the method comprises instructing the individual to ingest the capsule within 15 minutes of ingesting the delivery vehicle. In some embodiments, the capsule is incorporated within the food item. In some embodiments, the food item is configured to deliver the capsule incorporated therein to a specific portion of the gastrointestinal tract. Described herein is a method for performing a medical procedure on an individual, comprising: performing a capsule endoscopy on the individual; providing to the individual a composition comprising one or more food ingredients mixed with a laxative while the capsule endoscopy is performed; and performing a colonoscopy on the individual after the individual has ingested the composition. In some embodiments, a sufficient amount of the composition ingested by the individual produces a watery yellow stool with a light yellow effluent in the individual. In some embodiments, no additional bowel preparation is provided to the individual before the colonoscopy. In some embodiments, the one or more food ingredients comprise USP grade food ingredients. In some embodiments, the composition comprises a meal. In some embodiments, the composition comprises a solid food item. In some embodiments, the composition comprises a low residue food. In some embodiments, the solid food item comprises a total of 0-6 g fiber. In some embodiments, the solid food item comprises a total of 0.3-15 g Total Fats; 0-150 mg Cholesterol; 100-500 g of carbohydrates and/or 10-80 g of protein. In some embodiments, the solid food item provides a delivery vehicle for the laxative agent. In some embodiments, the composition eliminates or reduces side effects associated with administering to the subject food and laxative separately. In some embodiments, the side effects comprise one or more of dizziness, dehydration, nausea, vomiting, and taste fatigue. In some embodiments, the laxative is provided in an amount sufficient to induce the presence of the watery yellow stools with the light yellow effluent within about 6-8 hours after ingestion of the solid food item by the individual.

Also described herein is a delivery system comprising: a capsule for capsule endoscopy; a food item comprising one or more food ingredients; wherein the capsule is incorporated within the food item. In some embodiments, the quantity of the one or more food ingredients is such that when the delivery system is ingested by an individual, the one or more food ingredients will be digested to an extent that the capsule will be released within a duodenum of the individual. In some embodiments, the quantity of the one or more food ingredients is such that when the delivery system is ingested by an individual, the one or more food ingredients will be digested to an extent that the capsule will be released within a jejunum of the individual. In some embodiments, the quantity of the one or more food ingredients is such that when the delivery system is ingested by an individual, the one or more food ingredients will be digested to an extent that the capsule will be released within an ilium of the individual In some embodiments, the type of one or more food ingredients is such that when the delivery system is ingested by an individual, the one or more food ingredients will be digested to an extent that the capsule will be released within a duodenum of the individual. In some embodiments, the type of one or more food ingredients is such that when the delivery system is ingested by an individual, the one or more food ingredients will be digested to an extent that the capsule will be released within a jejunum of the individual.

### DETAILED DESCRIPTION

As used herein the term “capsule” when used by itself describes a capsule that is used in a capsule endoscopy procedure.

Described herein are compositions, methods, and kits for delivering a capsule in a capsule endoscopy procedure. Described herein are compositions and methods for preparing an individual for a colonoscopy following a capsule endoscopy.

In some embodiments, a food item comprises one or more edible ingredients. In some embodiments the one or more edible ingredients are USP Pharmacopeia-National Formulary grade ingredients. In some embodiments, the food item comprises a meal. In some embodiments, the food item comprises solid. In some embodiments, the food item comprises a liquid.

In some embodiments, a composition has the capsule used in capsule endoscopy incorporated therein. In some embodiments, the capsule used in capsule endoscopy is mixed together with one or more food ingredients to form a food item with the capsule incorporated therein. In some embodiments, the location of delivery of the capsule within the gastrointestinal tract is related to the rate of digestion of the composition in which the capsule is incorporated. For example, a composition comprising a thin edible layer takes relatively less time to digest and is digested in the proximal part of the intestinal tract releasing the capsule in the proximal part of the intestinal tract. For example, a composition comprising a thick edible layer takes relatively more time to digest and is digested in the distal portion of the digestive tract. Thus, by, for example, controlling the thickness of the composition, a point of release of the capsule within the gastrointestinal tract is controlled. Similarly, in some embodiments, the composition comprises of ingredients that are digested in the proximal portion of the gastrointestinal tract, and in some embodiments, the composition comprises ingredients that are digested in the distal portion of the gastrointestinal tract.

In some embodiments, a composition comprises an edible coating of the capsule used in capsule endoscopy.

In some embodiments, a composition for use in a capsule endoscopy procedure increases motility of the gastrointestinal tract to propel the capsule of the capsule endoscopy more rapidly through the gastrointestinal tract. In some embodiments, the composition increases gastrointestinal motility by stimulating normal digestive peristalsis. In some embodiments, the composition stimulates normal digestive peristalsis by promoting chewing in an individual. In some embodiments, the composition comprises a gum or other chewable. In some embodiments, the composition comprises a food item that stimulates normal digestive peristalsis by promoting digestion of the food item. In some embodiments, one or more food items comprise a kit, wherein the one or more food items are modular elements of the kit.

In some embodiments, the modular elements of the kit are ingested by an individual in a sequence after the individual has ingested the capsule. In this way, the sequentially ingested food items provided a sequential peristalsis effect that propels the capsule through the gastrointestinal tract. In some embodiments, the individual is instructed to ingest the modular elements of the kit in a specific order. In some embodiments, the individual is instructed to ingest the modular elements of the kit within a certain time from one another.

In some embodiments, peristalsis is promoted by a bowel motility promoting agent. Non-limiting examples of bowel motility promoting agents include Benzamide, Cisapride, Domperidone, Erythromycin, Itopride, Mosapride, Metoclopramide, Prucalopride, Renzapride, Tegaserod, Mitemcinal, Levosulpiride, and Cinitapride.

Described herein is a method for performing capsule endoscopy. In a first step, an individual undergoes a gastrointestinal preparation for an endoscopy procedure. In some embodiments, a first bowel preparation comprises a composition comprising a food item comprising one or more food ingredients mixed with a laxative. In some embodiments, the laxative comprises PEG. In some embodiments, the laxative comprises PEG 3350. Non-limiting examples of laxatives suitable for use include biscodyl, docusate, sodium phosphate, and senna. In some embodiments, the food item comprises a low fiber, low residue food item.

In a second step, a composition is provided to an individual to ingest together with the capsule used in a capsule endoscopy procedure. In some embodiments, a composition comprises a compound that propels a capsule through the gastrointestinal tract of an individual by stimulating peristalsis. In some embodiments, a composition comprises a compound for increasing gastrointestinal motility. In some embodiments, a composition comprises a compound that controls the release of a capsule in the gastrointestinal tract so that the composition determines the location or relative location within the gastrointestinal tract to which a capsule used in an endoscopy procedure is delivered. In some embodiments, a composition comprises a food item. In an alternative embodiment, the composition comprises the first bowel preparation so that the bowel preparation of the first step occurs with the ingestion of the composition rather than through a separate step.

In a third step, the individual to whom the composition and capsule were provided, ingests the composition and the capsule. In some embodiments, an instruction to ingest the composition and the capsule is provided to the individual and the individual ingests the composition and the capsule in response to the provided instruction. In some embodiments, the composition and the capsule are ingested either simultaneously or essentially simultaneously by the individual. In some embodiments, the capsule is incorporated into the composition thus both composition and capsule are ingested simultaneously when the composition is ingested. In some embodiments, the capsule is ingested before the composition is ingested. In some embodiments, the capsule is ingested up to 30 minutes before the composition is ingested. In some embodiments, the capsule is ingested up to 25 minutes before the composition is ingested. In some embodiments, the capsule is ingested up to 20 minutes before the composition is ingested. In some embodiments, the capsule is ingested up to 15 minutes before the composition is ingested. In some embodiments, the capsule is ingested up to 10 minutes before the composition is ingested. In some embodiments, the capsule is ingested up to 5 minutes before the composition is ingested. In some embodiments, the capsule is ingested up to 4 minutes before the composition is ingested. In some embodiments, the capsule is ingested up to 3 minutes before the composition is ingested. In some embodiments, the capsule is ingested up to 2 minutes before the composition is ingested. In some embodiments, the capsule is ingested up to 1 minute before the composition is ingested. In some embodiments, the composition is ingested before the capsule is ingested. In some embodiments, the composition is ingested up to 30 minutes before the capsule is ingested. In some embodiments, the composition is ingested up to 25 minutes before the capsule is ingested. In some embodiments, the composition is ingested up to 20 minutes before the capsule is ingested. In some embodiments, the composition is ingested up to 15 minutes before the capsule is ingested. In some embodiments, the composition is ingested up to 10 minutes before the capsule is ingested. In some embodiments, the composition is ingested up to 5 minutes before the capsule is ingested. In some embodiments, the composition is ingested up to 4 minutes before the capsule is ingested. In some embodiments, the composition is ingested up to 3 minutes before the capsule is ingested. In some embodiments, the composition is ingested up to 2 minutes before the capsule is ingested. In some embodiments, the composition is ingested up to 1 minute before the capsule is ingested.

In a fourth step, a capsule endoscopy is performed on the individual after the individual has ingested the composition and the capsule. In some embodiments, a kit is provided to an individual containing one or more compositions to be ingested sequentially by the individual throughout the capsule endoscopy procedure. In some embodiments, one or more compositions provided to the individual cleanse the gastrointestinal tract of the individual providing improved visualization for the capsule endoscopy procedure and also serving as a bowel preparation in the event that a colonoscopy is performed following the capsule endoscopy.

In some embodiments, in a fifth step an individual receives a second bowel preparation following the capsule endoscopy. In some embodiments, the second bowel preparation comprises a composition comprising one or more food ingredients mixed together with a laxative. In some embodiments the laxative comprises PEG. In some embodiments, the laxative comprises PEG 3350. Non-limiting examples of laxatives suitable for use include biscodyl, docusate, sodium phosphate, and senna. In some embodiments, the food item comprises a low fiber, low residue food item. In this embodiment, a colonoscopy is performed after the individual has completed the second bowel preparation. In some embodiments, the colonoscopy is performed within twenty-four hours of the capsule endoscopy. In some embodiments, the colonoscopy is performed within twelve hours of the colonoscopy.

Non-limiting examples of food items suitable for use with the compositions, methods, and kits described herein include solid foods, liquid foods, meals, bars, shakes, and edible powders.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for delivering a capsule comprising a camera to the gastrointestinal tract of an individual in order to perform a capsule endoscopy procedure, comprising:
providing a delivery vehicle to the individual to ingest with the capsule, wherein the delivery vehicle comprises a food item; and
performing the capsule endoscopy procedure after the individual has ingested the capsule and the delivery vehicle.

2. The method of claim 1, wherein the food item comprises a solid food.

3. The method of claim 1, wherein the delivery vehicle further comprises a bowel motility promoting agent, and wherein the bowel motility promoting agent is incorporated within the food item.

4. The method of claim 3, wherein the food item comprises one or more food ingredients mixed together with the bowel motility promoting agent.

5. The method of claim 3, wherein the bowel motility promoting agent comprises a laxative.

6. The method of claim 5, wherein the laxative comprises PEG 3350.

7. The method of claim 3, wherein the bowel motility promoting agent comprises one or more of Benzamide, Cisapride, Domperidone, Erythromycin, Itopride, Mosapride, Metoclopramide, Prucalopride, Renzapride, Tegaserod, Mitemcinal, Levosulpiride, and Cinitapride.

8. The method of claim 1, wherein performing the capsule endoscopy procedure occurs within one hour after the individual has ingested the capsule and the delivery vehicle.

9. The method of claim 1, comprising instructing the individual to ingest the capsule first and the delivery vehicle second.

10. The method of claim 9, comprising instructing the individual to ingest the delivery vehicle within 15 minutes of ingesting the capsule.

11. The method of claim 1, comprising instructing the individual to ingest the delivery vehicle first and the capsule second.

12. The method of claim 11, comprising instructing the individual to ingest the capsule within 15 minutes of ingesting the delivery vehicle

13. The method of claim 1, wherein the capsule is incorporated within the food item.

14. The method of claim 13, wherein the food item is configured to deliver the capsule incorporated therein to a specific portion of the gastrointestinal tract.

15. A method for performing a medical procedure on an individual, comprising:
performing a capsule endoscopy on the individual;
providing to the individual a composition comprising one or more food ingredients mixed with a laxative while the capsule endoscopy is performed; and
performing a colonoscopy on the individual after the individual has ingested the composition.

16. The method of claim 15, wherein a sufficient amount of the composition ingested by the individual produces a watery yellow stool with a light yellow effluent in the individual.

17. The method of claim 15, wherein no additional bowel preparation is provided to the individual before the colonoscopy.

18. The method of claim 15, wherein the one or more food ingredients comprise USP grade food ingredients.

19. The method of claim 15, wherein the composition comprises a meal.

20. The method of claim 15, wherein the composition comprises a solid food item.

21. The method of claim 20, wherein the composition comprises a low residue food.

22. The method of claim 20, wherein the solid food item comprises a total of 0-6 g fiber.

23. The method of claim 20, wherein the solid food item comprises a total of 0.3-15 g Total Fats; 0-150 mg Cholesterol; 100-500 g of carbohydrates and 10-80 g of protein.

24. The method of claim 20, wherein the solid food item provides a delivery vehicle for the laxative agent.

25. The method of claim 15, wherein the composition eliminates or reduces side effects associated with administering to the subject food and laxative separately.

26. The method of claim 25, wherein the side effects comprise one or more of dizziness, dehydration, nausea, vomiting, and taste fatigue.

27. The method of claim 26, wherein the laxative is provided in an amount sufficient to induce the presence of the watery yellow stools with the light yellow effluent within about 6-8 hours after ingestion of the solid food item by the individual.

28. A delivery system comprising:
a capsule for capsule endoscopy;
a food item comprising one or more food ingredients;
wherein the capsule is incorporated within the food item.

29. The delivery system of claim 28, wherein the quantity of the one or more food ingredients is such that when the delivery system is ingested by an individual, the one or more food ingredients will be digested to an extent that the capsule will be released within a duodenum of the individual.

30. The delivery system of claim 28, wherein the quantity of the one or more food ingredients is such that when the delivery system is ingested by an individual, the one or more food ingredients will be digested to an extent that the capsule will be released within a jejunum of the individual.

31. The delivery system of claim 28, wherein the quantity of the one or more food ingredients is such that when the delivery system is ingested by an individual, the one or more food ingredients will be digested to an extent that the capsule will be released within an ilium of the individual.

32. The delivery system of claim 28, wherein the type of one or more food ingredients is such that when the delivery system is ingested by an individual, the one or more food ingredients will be digested to an extent that the capsule will be released within a duodenum of the individual.

33. The delivery system of claim 28, wherein the type of one or more food ingredients is such that when the delivery system is ingested by an individual, the one or more food ingredients will be digested to an extent that the capsule will be released within a jejunum of the individual.
